(19) United States
(12) Patent Application Publication
(10) Pub. No.: US 2006/0135444 A1
(43) Pub. Date: Jun. 22, 2006

(54) Combination of flavonoid and procyanidin for the reduction of the mammalian appetite

(76) Inventors: Arie Gijsbert Nieuwenhuizen, Utrecht (NL); Katrien Maria Jozefa Van Laere, Heteren (NL); Rene John Raggers, Amsterdam (NL)

Correspondence Address: Young & Thompson, Second Floor, 745 South 23rd Street, Arlington, VA, 22202, US

(21) Appl. No.: 10/535,258

(22) PCT Filed: Mar. 18, 2003
(86) PCT No.: PCT/NL03/00198

§ 371 Date: Jan. 18, 2006

Related U.S. Application Data
(63) Continuation of application No. 10/295,987, filed on Nov. 18, 2002, now abandoned.

(51) Int. Cl. A61K 31/522; A61K 31/7048; A61K 31/353

(52) U.S. Cl. 514/27; 514/263.31; 514/456

(57) ABSTRACT

A nutritional composition suitable for reducing appetite, a method for the treatment and/or prevention of overweight and a method for the reduction of a mammalian appetite. The weight reduction and/or appetite reduction is achieved by administration of procyanidin and a flavonoid selected from the group consisting of chrysin, flavone, precursors of these flavonoids that are convertible into the one of these flavonoids by gastrointestinal hydrolytic cleavage and mixtures thereof.

The invention relates to a method for the prevention or treatment of overweight, the method comprising the administration of flavonoids and procyanidins.

### BACKGROUND OF THE INVENTION

Overweight and obesity are major problems within the Western community. Due to increased consumption, decreased exercise and changes in the nutritional value of foodstuffs, many humans and companion animals are suffering from overweight or have difficulty maintaining a desirable weight. Many methods have been proposed to solve this problem, for example, via the administration of functional ingredients (e.g. nutritional supplements) which facilitate the reduction of overweight.

In effect, overweight is caused by the ingestion of excess calories. Calories are for example ingested via high caloric meals. Within the art, several strategies are known for reducing caloric intake. These strategies include for example replacing the high caloric meals for low caloric meals (e.g. meal replacers); the ingestion of medicaments that reduce the absorption of high caloric components from the meal (e.g. lipase inhibitors); and the administration of appetite reducing agents.

Currently,-known appetite reducing agents have the disadvantage of causing severe side effects. Sibutramine is an appetite suppressant that is proposed to work via norepinephrine and serotonergic mechanisms in the brain. The drug has been described to have side effects, including high blood pressure, headache, dry mouth (which may increase the risk for dental disease), constipation and decreased sleep. Other reported side effects include: increased heart rate, dizziness, flushing, sweating, nausea. Presently, sibutramine is the only appetite suppressant that has been approved by the FDA for long-term use in a method for reducing overweight.

The present inventors have surprisingly found that chrysin (CAS Registry Number 480-40-0) as well as flavone (CAS Registry Number 525-82-6) are capable of reducing the appetite when administered orally, as described in the Applicant US co-pending application “METHOD FOR THE REDUCTION OF THE MAMMALIAN APPETITE” filed 18 Nov. 2002 with U.S. Ser. application No. 10/295987 and incorporated herein by reference.

Typically, Chrysin (5,7-dihydroxyflavone) is a flavonoid that is advertised to promote muscle growth. Furthermore, chrysin has been described to possess anxiolytic properties (i.e. anxiety reducing properties) without exhibiting a sedative effect as given in U.S. Pat. No. 5,756,538. Chrysin has also been described to treat any condition of elevated levels of unconjugated bilirubin in adults or children, such as Gilbert's syndrome or liver cirrhosis as given in WO 01/58410.

WO 99/22728 relates to compounds that inhibit 5 alpha-reductase. The compounds are used to treat prostate cancer, breast cancer, obesity, skin disorders and baldness. From Table 1 in the document it is apparent that chrysin is unsuitable as a compound to inhibit 5 alpha reductase, hence unsuitable to treat prostate cancer, breast cancer, obesity, skin disorder and baldness.

The present inventors have also found that procyanidins are capable of reducing the mammalian appetite when administered orally, as described in the Applicant co-pending application PCT/NL 02/00607 and incorporated herein by reference.

Procyanidins are known and used for their antioxidant properties. Still, these have also been described for their antiobesic properties, i.e. the intestinal lipase and carbohydrase reducing activity.

EP815857 describes an antiobestic agent comprising as the active ingredient tamarind seed coat extracts (procyanidin). According to this application, the tamarind seed coat extract or procyanidin can act as a carbohydrase inhibitor, blood sugar increase inhibitor, monosaccharide absorption inhibitor, cholic acid adsorptive excretion promoter, cholesterol lowering agent, blood triglyceride lowering agent and lipase inhibitor.

WO 00/54610 describes a food complement for dietetic and/or cosmetic purposes, containing anti-lipase properties, for oral administration. Said food complement is characterized in that it comprises a grape extract rich in or enriched with polyphenols.

The present inventors have now surprisingly found that a combination of flavone and/or chrysin combined with procyanidin is particularly suitable for reducing mammalian appetite, and can be suitably used in a method for the treatment of overweight. Reduction of appetite results in a reduced caloric intake.

Grape seed generally contains a minor percentage procyanidins. Further, combinations of chrysin and grape seed are known in the art, for example from Vitagrowth IV™, a product advertised as an “Advanced Herbal Solution for Incontinency”. However, such product only contains minor quantities of flavone, chrysin and procyanidin. Consequently, the product is deemed to comprise insufficient procyanidins and/or chrysin to provide the appetite reducing effects when combined with chrysin.

### SUMMARY OF THE INVENTION

In one aspect of the present invention, there is provided a nutritional composition suitable for enteral administration comprising per dosage:
a. between 5 mg and 10 grams of a flavonoid selected from the group consisting of chrysin, flavone, precursors of these flavonoids that are convertible into the one of these flavonoids by gastrointestinal hydrolytic cleavage and mixtures thereof; and
b. between 10 mg and 12.5 g procyanidin.

In a further aspect the present invention relates a method for the treatment and/or prevention of overweight in a mammal, said method comprising administering to the mammal:
a. between 0.01 mg and 250 mg flavonoid per kg body weight of the mammal, said flavonoid being selected from the group consisting of chrysin, flavone, precursors of these flavonoids that are convertible into the one of these flavonoids by gastrointestinal hydrolytic cleavage and mixtures thereof; and
b. between 0.1 and 100 mg procyanidin per kg body weight of the mammal.

In still a further aspect the present invention relates a method for the reduction of appetite in a mammal, said method comprising administering to the mammal:
a. between 0.01 mg and 250 mg flavonoid per kg body weight of the mammal, said flavonoid being selected from the group consisting of chrysin, flavone, precursors of these flavonoids that are convertible into the one of these flavonoids by gastrointestinal hydrolytic cleavage and mixtures thereof; and
b. between 0.1 and 100 mg procyanidin per kg body weight of the mammal.

A further aspect of the present invention relates to the provision of an article of manufacture comprising a packaging with one or more of the above described dosages, in which said article bears a label indicating that the contents should be consumed by a human desiring to lose weight.

### DETAILED DESCRIPTION OF THE PREFFERED EMBODIMENTS

Flavonoids

The present inventors surprisingly found that chrysin and flavone are capable of reducing the mammalian appetite. Hence, the present method comprises the administration of an effective amount of a flavonoid selected from-the group consisting of chrysin, flavone, precursors of these flavonoids that are convertible into the one of these flavonoids by gastrointestinal hydrolytic cleavage and mixtures thereof. Preferably, the precursors of the flavonoids that are convertible into one of these flavonoids by gastrointestinal hydrolytic cleavage are selected from the group consisting of glycosides, rutinosides, glucuronoside, gentobioside and methyl ethers, more preferably glycosides.

For the purpose of the present invention, by the term “glycosides” it is meant glycosides of chrysin and/or glycosides of flavone.

Advantageously chrysin or a precursor thereof such as a glycoside thereof is used. Preferred glycosides of chrysin are selected from the group consisting of chrysin-5-O-glycoside and chrysin-7-O-glycoside. Most preferably chrysin is used.

The precursor of the flavonoids that are convertible into flavone or chrysin by gastrointestinal hydrolytic cleavage is preferably also converted into flavone or chrysin under (in vitro) conditions that simulate the gastrointestinal condition(s), preferably human gastrointestinal conditions. This typically involves incubating the precursor in a medium that simulate the human gastrointestinal conditions and observe if chrysin or flavone is formed. More preferably, the precursor is converted into chrysin or flavone under acidic conditions and/or by the action of one or more gastrointestinal enzymes, normally present in the gastrointestinal tract.

Dosage Flavonoid

In order to achieve significant weight reduction and/or appetite reducing effect, it is desirable to administer at least 0.01 mg, preferably at least 0.1 mg, even more preferably at least 1 mg, most preferably at least 2 mg of the flavonoid per kg of body weight. Preferably the amount is administered on a daily basis, more preferably per serving. Preferably, the total amount of the present flavonoid does not exceed 250 mg per kg of body weight

A dosage, suitable for use in the present method, preferably contains at least 10 mg, more preferably at least 50 mg, even more preferably at least 100 mg, most preferably at least 200 mg of the flavonoid. To prevent undesirable side effects, the dosage preferably contains less than 10 g, more preferably less than 5 g, even more preferably less than 2.5 g of the flavonoid.

For a human adult, the present method preferably comprises the administration of a dosage containing between 10 mg and 10 grams, more preferably between 50 mg and 5 grams, even more preferably between 100 mg and 2.5 grams of the flavonoid

Sources of the Flavonoids

In a particularly preferred embodiment, the flavonoid, is provided by plant material containing the flavonoid, more preferably in the form of a plant isolate containing the flavonoid.

The term “plant isolate” as referred to herein, encompasses any fraction that can be obtained from a plant material by means of isolation techniques known in the art, e.g. extraction, distillation, squeezing etc. and that has an increased flavonoid content compared to the dry plant raw material. Preferably the plant material contains at least 1 wt. %, more preferably at least 5 wt. %, even more preferably at least at least 10 wt. %, most preferably at least 50 wt. % flavonoid based on the total dry weight of the plant material. Usually, the flavonoid content of the plant material does not exceed 99.5 wt. % based on the total dry weight of the plant raw material. Advantageously, synthetic chrysin is used.

According to a preferred embodiment the present flavonoid, preferably chrysin, is provided in the form of a plant material or extract from a plant selected from the group consisting of Pinus aristata, Prunus domestica, Ulmus sieboldiana, Fluorensia resinosa, Oroxylum indicum, Scutellaria spp., Passiflora spp. and mixtures thereof, more preferably in the form of a plant selected from the group consisting of Pinus aristata, Prunus domestica, species of Passiflora and mixtures thereof, most preferably selected from plant material or isolate from a plant selected from the group consisting of Passiflora alata, Passiflora incarnata, Passiflora coerulea and mixtures thereof.

Flavonoid Content

Advantageously, the present method comprises the administration of a serving or dosage containing at least 1 wt. %, more preferably at least 5 wt. %, even more preferably at least 10 wt. %, most preferably at least 25 wt. % of the present flavonoid based on the total dry weight of the dosage. Preferably the flavonoid content of the dosage does not exceed 90 wt. % flavonoid, more preferably does not exceed 50 wt. % based on the total dry weight of the dosage.

Procyanidin

Procyanidins have been known and used especially for their antioxidant properties and their carbohydrase inhibitory effect. It has surprisingly been found by the present inventors that procyanidins have appetite reducing properties, when administered to a mammal in a therapeutically effective amount.

Procyanidins are members of the proanthocyanidins group. Proanthocyanidins involve procyanidin, prodelphinidin, propelargonidin, proguibourtinidin, profisetinidin, prorobinetinidin, proteracacidin, promelacacidin, proapigeninidin, proluteolinidin and all of the stereoisomers thereof. The procyanidin used in the present invention is a polymer, comprising 2 or more units of one or more of the monomers as shown in the following formula:

[image]

wherein:

a. R₁═OH,R₂═H,R₃═H; or
b. R₁═H,R₂═OH,R₃═H; or
c. R₁βgallic acid ester, R₂═H, R₃═H; or
d. R₁═H, R₂=gallic acid ester, R₃═H

According to a preferred embodiment the procyanidin is mainly present as B-type polymers, which have a single interflavanoid bond (in contrast to A-type procyanidin polymers, which have a second interflavanoid bond). Even more preferably at least 70 mol % of the procyanidin polymers are present as B-type polymers.

Procyanidin: Source

Procyanidins can be easily obtained from various sources. Preferably the procyanidins are obtained from a natural, more preferably a plant source. Preferably the procyanidins are obtained from a plant source selected from the group consisting of grape, pine, cocoa, tamarind, tomato, peanut, almond, apple, cranberry, blueberry and mixtures thereof, especially from the group consisting of pine bark, grape seed, tamarind seed husk, cocoa bean, apple peel, apple pericarp and mixtures thereof and most preferably from grape seed and/or pine bark. Advantageously, procyanidins from grape seed are used

Procyanidins: Extract

Typically procyanidins from plant sources comprise very low levels of procyanidins. Consequently, in order to accomplish the desired appetite reducing effect, vast amounts of the raw plant material would have to be consumed. The use of an extract of one of the procyanidin sources may avoid such discomfort. An additional advantage of the use of a more concentrated form of procyanidins, e.g. in the form of an extract, resides in the fact that procyanidins may be administered without co-administering a significant amount of caloric plant material. More importantly, however, the inventors believe that the use of compositions having an increased weight percentage of procyanidins provides an increased appetite reducing effect compared to procyanidin containing compositions having a lower weight percentage procyanidins.

Preferably, the procyanidin containing plant extract used in the current invention comprises at least 5 wt. %, more preferably at least 10 wt. %, even more preferably at least 25 wt. %, most preferably at least 50 wt. %, especially at least 75 wt. % procyanidin based on the dry weight of the extract. The term “extract” as used in the present invention refers to an isolate that has been obtained by means of solvent extraction.

Procyanidin: Dosage

In accordance with a preferred embodiment, a dosage comprising between 0.1 and 100 mg procyanidin per kg body weight should be administered to a mammal. More preferably the method comprises the administration of between 0.5 and 75 mg, even more preferably between 0.75 and 50 mg, most preferably between 1 and 20 mg, especially between 2 mg and 10 mg procyanidin per kg body weight. For human subjects above the age of about 10 years, the present dosage contains between 10 mg and 12.5 g, preferably between 25 mg and 10 g, more preferably between 50 mg and 5 gram, more preferably between 100 mg and 3 gram, most preferably between about 200 mg and 2 gram, especially between 250 mg and 1 gram procyanidin.

According to a preferred embodiment, the appetite reducing composition comprising procyanidin, comprises at least 1 wt. % procyanidin based on dry weight, more preferably at least 2 wt. %, even more preferably at least 5 wt. %, most preferably at least 8 wt. %, especially at least 12.5 wt. %. Preferably the procyanidin content of the dosage does not exceed 90 wt. % procyanidin, more preferably does not exceed 50 wt. % based on dry weight.

Nutritional Composition

In a further aspect, the present invention provides a nutritional composition for enteral administration comprising per serving: a) between 5 mg and 10 grams of a flavonoid selected from the group consisting of chrysin, flavone, precursors of these flavonoids that are convertible into the one of these flavonoids by gastrointestinal hydrolytic cleavage and mixtures thereof ; and b) between 10 mg and 12.5 g procyanidin.

Preferably the present nutritional composition is in the form of a unit dosage. Whenever the term dose or dosage is used-within this disclosure, any dosage form is encompassed which can be administered, preferably orally, within a fairly narrow time span. Whenever reference is made to a certain quantity that is administered per dose or dosage, said quantity is preferably administered within one hour, more preferably within 15 minutes, even more preferably within 5 minutes.

Flavonoid and Procyanidin: Synergism

It is the inventors belief that the procyanidin combined with one or more of the present flavonoids synergistically reduce the appetite of a mammal. The effect is deemed to be synergistically increased if sufficient amounts of the flavonoid, preferably chrysin, and procyanidins are administered simultaneously.

Treatment and Prevention of Overweight

The present invention provides a method for reducing or preventing overweight or obesity, and a method for the reduction the appetite in a mammal. The term overweight as used in the present invention refers to a bodyweight that is above the desired bodyweight. The present method is particularly suitable for humans, especially most advantageous for human subjects who have a body mass index above 25.

The present invention also provides an article of manufacture comprising packaged unit dosages according to the present invention, in which said article bears a label indicating the contents should be consumed by a human desiring to lose weight. Hence, packaged nutritional supplements and dietary products, which have been provided with labels that explicitly or implicitly direct the consumer towards the use of said supplement or product in accordance with one or more of the above or below purposes, are encompassed by the present invention. Such labels may for example make reference to the method for the treatment of overweight by incorporation of terminology like “slim”, “lean”, “weight reduction”, “appetite reduction”, “satiety” and the like. The overweight reducing properties of the product may be indicated via indicia such as pictures, drawings and other indicia from which a consumer can conclude that the product aims to treat or prevent overweight.

The present invention preferably does not relate to the use of chrysin for counteracting side effects of the androgens, anabolic steroids or prohormones. Androgens, anabolic steroids and prohormones are often used to increase muscle mass, and thus body weight. In a preferred embodiment the present method does not comprise the administration of a member selected from the group consisting of an androgens, anabolic steroids and prohormones, i.e. comprises the administration of a dosage free of androgens, anabolic steroids and prohormones. In other words, the present method preferably does not comprise the administration of any androgens, anabolic steroids or prohormones. The term “prohormones” as used in the present invention relates to metabolic precursors which raise the level of the male hormones testosterone and/or 19-nortestosterone in vivo, preferably to those ingredients that are metabolic precursors of hormones.

Thermogenic Ingredient

Due to the appetite reducing effects of the present combination of flavonoid and procyanidin, the in vivo nutrient availability becomes limited. After some time, this may translate into a hunger feeling. Thermogenic ingredients are capable of increasing the in vivo availability of free fatty acids from in vivo stored fat (e.g. triglycerides stored in adipose tissue. Co-administration of the thermogenic ingredient together with the present combination of flavonoid and procyanidin is believed to prolong the appetite reducing effects of the flavonoid and procyanidin. Additionally, in a method for treating overweight and reducing adiposity it is especially advantageous to preferentially oxidate fatty acids, as it reduces fat stores. Hence, the flavonoid, procyanidin and thermogenic ingredient are deemed to synergistically reduce overweight.

Preferably the thermogenic ingredient is selected from the group consisting of caffeine, synephrine, ephedrine, Bacopa plant material, Paullinia cupana and Ilex paraguariensis, more preferably is Ilex paraguariensis.

The thermogenic ingredient is preferably administered in a daily amount of between 0.1 mg and 25 mg per kg of body weight. According to a particularly preferred embodiment the thermogenic ingredient is administered in a daily amount between 0.5 and 15 mg, more preferably between 1 and 10 mg thermogenic ingredient per kg body weight.

The dosage according to the present invention preferably comprises between 5 and 4000 mg thermogenic ingredient. More preferably, a dosage comprises between 25 and 2000 mg, even more preferably between and 50 and 1000 mg, most preferably between 100 and 500 mg thermogenic ingredient.

Advantageously, the present method comprises the co-administration of caffeine. The caffeine used in the present invention may be naturally, semi-synthetically or synthetically derived. According to a preferred embodiment naturally derived caffeine is used. Preferred sources of naturally derived caffeine include herbal extracts comprising between 5 and 95% caffeine based on the dry weight of the herbal extract, more preferably between 10 and 50%. A preferred source of natural caffeine is a plant material selected from the group consisting of Paullinia cupana and Ilex paraguariensis. Most preferably Ilex paraguariensis is used.

The caffeine is preferably administered in a daily amount of between 0.1 mg and 25 mg per kg of body weight. According to a particularly preferred embodiment the caffeine is administered in a daily amount between 0.5 and 15 mg, more preferably between 1 and 10 mg caffeine per kg body weight.

The dosage according to the present invention preferably comprises between 5 and 4000 mg caffeine. More preferably, a dosage comprises between 25 and 2000 mg, even more preferably between 50 and 1000 mg, most preferably between 100 and 500 mg caffeine.

Fibers

In a preferred embodiment, the present invention comprises the coadministration of fibers. The fibers further increase the satiation, by providing a feeling of fullness which is probably caused by the a reduced rate of stomach emptying.

The fiber used in accordance with the present invention is preferably selected from the group consisting of gum arabic, sodium carboxymethylcellulose, methylcellulose, guar gum, gellan gum, locust bean gum, konjac flour, hydroxypropyl methylcellulose, tragacanth gum, karaya gum, gum acacia, chitosan, arabinoglactins, glucomannan, xanthan gum, alginate, pectin, β-glucans, carrageenan and psyllium, more preferably from the group consisting of glucomannan, guar gum, gellan gum and locust bean gum.

The fiber is preferably administered in a daily amount of between 0.5 mg and 100 mg per kg of body weight. According to a particularly preferred embodiment the fiber is administered in a daily amount between 2 and 75 mg, more preferably between 2.5 and 50 mg fiber per kg body weight.

The dosage according to the present invention preferably comprises between 5 mg and 15 grams fiber. More preferably, a dosage comprises between 50 mg and 10 grams, even more preferably between and 100 mg and 5000 mg, most preferably between 250 mg and 2500 mg fiber.

Reduction of Appetite

The present invention is especially aimed at the reduction or prevention of appetite and/or feelings of hunger. The method according to the invention can, for example, be used in a method for inducing satiety, inducing satiation, satisfying hunger or reducing craving urges.

Generally, an individual's feelings and sensations between the start of a first meal and the next meal go through different phases. A set of sensations is usually discriminated within the art. If satiety is evaluated, several phases can be used to express the satiety after a meal. These can be termed very full, full, appetite and hungry. Preferably the present invention combination of the flavonoid and procyanidin is administered in the phases appetite, hunger or at the end of the full phase, more preferably in the appetite or hunger phase.

Preferably, the flavonoid and procyanidin is administered about 1-8 hours, more preferably about 2-6 hours after consumption of a meal. Typically the procyanidin and flavonoid is administered between 1 hour after one meal and 1 hour prior to the next meal. In a further aspect, the procyanidin and flavonoid can be taken shortly before the meal or even during a meal, for example when the meal is expected to provide insufficient satisfaction. This may occur when the individual is subjected to a weight loss program. Hence, the nutritional composition can be advantageously used in a method for the reduction of the adverse side effects experienced during a weight loss program, i.e. administered to subjects participating in a weight loss program.

Additionally, appetite reducing agents are useful in several other applications. The present invention combination of flavonoid and procyanidin can for example be used to provide comfort to subjects having limited access to foodstuffs, such as for example military personal during a long mission.

Administration

According to a preferred embodiment the present method comprises the enteral, more preferably the oral administration of the flavonoid and procyanidin. Particularly suitable is the administration of a dosage or serving containing both the flavonoid and the procyanidin.

The dosage used in the present method can be applied in any suitable form, such as bars, pills, capsules, gels, liquid, etc. However, it is preferably provided in the form of a pill, tablet or capsule. Preferably a dosage does not consist of more than 3 tablets, capsules or pills, even more preferably consists of a single pill, capsule or tablet. Advantageously, at least two dosages, more preferably at least three dosages, are administered in one day. In the present method a daily dosage of the preparation as used in the present invention can include one or more pills, tablets or capsules. Preferably a daily dosage consists of 1 to 6 pills, tablets or capsules.

A dosage is preferably in a solid or semisolid form, more preferably in a form selected from the group consisting of pills, capsules, tablets, caplet, microparticles and microspheres. The solid or semisolid dosage form preferably has a weight between 0.1 and 30 grams, more preferably between 0.2 and 10 grams.

For easy administration, the dosage preferably contains a pharmaceutically acceptable carrier. These carriers may be selected from sugars, starches, cellulose and its derivatives, malt, gelatine, talc, calcium sulfate, vegetable oils, synthetic oils, polyols, alginic acid, phosphate buffered solutions, emulsifiers, isotonic saline, and pyrogen-free water

The dosage preferably has a caloric value below 100 kcal, more preferably below 50 kcal even more preferably below 10 kcal. A dosage preferably has a weight between 0.2 and 4 grams, even more preferably between 0.5 and 3 grams.

The following are non-limiting examples illustrating the present invention:

### EXAMPLES

### Example 1

### Weight Loss Supplement for Humans

Soft gelatin capsule containing,
800 mg Vitis vinifera extract (comprising 85 wt. % procyanidins); and
200 mg chrysin (min 99%, Pharm Chemical, Shanghai, China)

### Example 2

### Appetite Reducing Supplement for Humans

Soft gelatin capsule containing,
400 mg pycnogenol (comprising 65 wt. % procyanidins);
800 mg chrysin (min 99%, Pharm Chemical, Shanghai, China); and
200 mg excipient

### Example 3

### Anti-Craving Supplement for Humans

A beverage containing per 200 ml serving
800 mg Vitis vinifera extract (comprising 85 wt. % procyanidins)
250 mg Gymema sylvestre extract (comprising 25 wt. % gymnemic acid); and
200 mg chrysin (min 99%, Pharm Chemical, Shanghai, China)

### Example 4

### Appetite Reducing Bar for Children

Cereal bar containing:
50 mg procyanidins in the form of apple chunks;
30 gram oat bran;
50 mg chrysin (min 99%, Pharm Chemical, Shanghai, China); and
200 mg guar gum

### Example 5

### Appetite Reducing Supplement for Humans

Soft gelatin capsule containing,
200 mg Vitis vinifera extract (comprising 25 wt. % procyanidins);
200 mg chrysin (min 99%, Pharm Chemical, Shanghai, China); and
100 mg caffeine

1-15. (canceled)

16. A method for treating and/or preventing overweight in a mammal, comprising administering to said mammal a preparation which comprises:
a. between 0.01 mg and 250 mg of said flavonoid per kg body weight of said mammal, said flavonoid being selected from the group consisting of chrysin, flavone, a flavonoid precursor that is convertible into a flavonoid by gastrointestinal hydrolytic cleavage, and mixtures thereof; and
b. between 0.1 and 100 mg procyanidin per kg body weight of said mammal.

17. The method according to claim 16, wherein said flavonoid is chrysin.

18. The method according to claim 16, wherein said preparation contains at least 1 wt % of flavonoid based on the total dry weight of the preparation.

19. The method according to claim 16, wherein said preparation contains at least 1 wt % of procyanidin based on the total dry weight of the preparation.

20. The method according to claim 16, wherein said preparation is administered orally.

21. The method according to claim 16, wherein said preparation further comprises a thermogenic ingredient.

22. The method according to claim 21, wherein said thermogenic ingredient is caffeine.

23. The method according to claim 16, wherein said preparation further comprises between 0.5 mg and 100 mg fiber per kg body weight of the mammal.

24. A nutritional composition suitable for enteral administration to reduce appetite comprising:
a. between 5 mg and 10 grams of a flavonoid selected from the group consisting of chrysin, flavone, a flavonoid precursor that is convertible into a flavonoid by gastrointestinal hydrolytic cleavage, and mixture thereof; and
b. between 10 mg and 12.5 g procyanidin.

25. The nutritional composition according to claim 24, wherein said flavonoid component is chrysin.

26. The nutritional composition according to claim 24, further comprising a thermogenic ingredient.

27. The nutritional composition according to claim 26, wherein said thermogenic ingredient is caffeine.
